(19) United States
(12) Patent Application Publication
(10) Pub. No.: US 2018/0049941 A1
(43) Pub. Date: Feb. 22, 2018

(54) MASSAGE ROLLER

(71) Applicants: Michael Venezia, El Segundo, CA (US); Christa Pryor, Playa del Rey, CA (US); Vince Guagliano, Rolling Hills Estates, CA (US)

(72) Inventors: Michael Venezia, El Segundo, CA (US); Christa Pryor, Playa del Rey, CA (US); Vince Guagliano, Rolling Hills Estates, CA (US)

(21) Appl. No.: 15/683,768

(22) Filed: Aug. 22, 2017

Related U.S. Application Data
(60) Provisional application No. 62/377,940, filed on Aug. 22, 2016.

(51) Int. Cl. A61H 15/02; A61N 5/06

(52) U.S. Cl. CPC A61H 15/02; A61H 2015/0014; A61N 5/06

(57) ABSTRACT

Provided is a massage roller or ball comprising: a) a rigid cylindrical or spherical body; b) a sheet of a heating pad placed over the cylindrical body; c) a rechargeable battery placed inside of the cylindrical or spherical body; wherein current from the battery makes the heating pad increase in temperature. Provided is a massage roller comprising: a) a rigid cylindrical body; b) two end caps, with each end cap placed at one end of the cylindrical body; c) a sheet of foam placed over the cylindrical body; d) a sheet of woven carbon fiber placed over the foam, the sheet of carbon having two conductors that do not touch each other; e) a cylindrical shell placed inside of the body; f) a rechargeable battery placed inside of the shell; g) two lead wires carrying current from the battery to the conductor, each wire connected to one of the conductors; wherein current from the battery heats the heating pad.

### CROSS-REFERENCE

The present application claims the benefit of U.S. provisional patent application No. 62/377,940, filed on Aug. 22, 2016, which is incorporated herein by reference in its entirety.

### BACKGROUND OF THE INVENTION

Stretching is noted to be beneficial to those who do and who do not participate in exercise. In many instances, stretching is not enough to alleviate fasciculations, adhesions or tightness (sick tissue), as stretching only addresses healthy tissue, but neglect sick tissue. Several techniques have been used to reduce sick tissues. Elongating tissue with compression has proven effective in reducing sick tissue. There is a need in the art for an apparatus to be used in medical and exercise applications for stretching, massage, posture, and balance.

### SUMMARY OF INVENTION

Provided is a massage roller or a ball comprising: a) a rigid cylindrical or spherical body; b) a sheet of a heating pad placed over the cylindrical or spherical body; c) a rechargeable battery placed inside of the cylindrical or spherical body; wherein current from the battery makes the heating pad increase in temperature. The heating pad can get heated to a temperature of 100 to 150 Fahrenheit. A sheet of foam can be placed in between the heating pad and the cylindrical body. The roller can comprise two end caps, each end cap placed at one end of the cylindrical or spherical body. The end caps can have ventilation openings.

The massage roller can comprise a fan inside of the cylindrical or spherical body, the fan configured to move air through the vents of one of the end caps. The roller can further comprise a shell for placing electronic components (Circuit board, resistors, capacitors). The electronic housing is placed in the cylindrical or spherical body. The end cap can comprise a plug configured to receive a wire that carries electricity. The roller can further comprise an on/off switch placed on one of the end caps. The roller can further comprise a laminate on top of the heating pad. The laminate can be polyurethane laminate fabric. The heating pad can be made from a non-metal material and/or composite material, such as a carbon based material. The carbon based material can be a sheet of woven carbon fiber. The heating pad can have two strips of conductors that do not touch each other, each strip in contact with a lead wire. The heating pad can be rectangular in shape, and further comprising placing two strips of conductors in parallel configuration along opposite sides of the pad. The roller can further comprise a power switch on one of the end caps and a level of heating switch on another end cap. The heating pad can be in a form of a removable sleeve with or without a cover. Provided is a mat (like a yoga mat) with one or more above components, such as a layer of foam, a layer of heating pad on top of the foam, and a top cover, the mat in electronic communication with a rechargeable battery. The mat can be a portable mat.

Provided is a massage roller or massage ball comprising: a) a rigid cylindrical or spherical body; b) two end caps, with each end cap placed at one end of the cylindrical body; c) a sheet of foam placed over the cylindrical body; d) a sheet of woven carbon fiber placed over the foam, the sheet of carbon having two conductors that do not touch each other; e) a cylindrical or spherical shell placed inside of the body; f) a rechargeable battery placed inside of the shell; g) two lead wires carrying current from the battery to the conductor, each wire connected to one of the conductors; wherein current from the battery heats the heating pad. The shell can be removable. The massage roller can further comprise a fan for moving air out of the shell from a plurality of vents on at least one of the end caps.

The present invention is directed to a thermal and FIR (Far Infra Red) therapy sleeve by which it is to be placed over a massage roller/hard-cylindrical tube, sphere or human extremity. The hard cylindrical tube is to be made of a acrylonitrile butadiene-styrene (abs) due to its rigidity and ability to sustain impact, as well as its inability to form hydrochloric acid in the advent of fire as opposed to its PVC counterpart. The tube is hollow in its incarnation and will house a plastic molded insert by which the circuitry and power source will be mounted. The power source is a rechargeable battery, as described below, will supply power to the heating element and user display(s). Affixed on top of the tube will be the insulated heating element (described below).

The heating element is formed of carbon fiber with two lead electrodes conducting electricity through fabric allowing for constant flow of current. This, in turn, provides for consistent heat throughout element. The heating element is surrounded by a thermal bonding film on both sides. The “back” of the covered heating element is affixed to the foam roller with heat resistant adhesive. Foam affixed to the roller acts as an insulator from exogenous heat loss to the environment. The therapeutic heat side of the heating element covered in thermal bonding film is covered with a waterproof, washable material which can be either wiped down, or removed and washed to prevent transmission of disease and dirt.

The battery and heating element are controlled by a voltage/display controller printed circuit board (V-PCB) and a charging circuit printed circuit board (C-PCB). The V-PCB controls voltage output of the battery and display functions for the user. The C-PCB's function is to charge the Li Ion battery. It receives AC current from an attached plug which is ultimately plugged into a wall receptacle. Charging is permitted when the unit is off to limit over heating of the battery.

The battery, C-PCB and V-PCB are mounted on the plastic molded insert which is ultimately mounted in the cylinder. The plastic molded mount is designed to house the components such that they are balanced for consistent rolling and absorption of exogenous shock, i.e. a fall or drop. The components are strategically placed for heat conduction.

End caps are placed on either side of the roller to house the charging apparatus, as well as the user display and controls.

Mounted to an end cap is a fan to facilitate cross ventilation, ultimately conducting heat out of the roller, thus preventing over-heating.

It is accordingly an object of the present invention to provide a thermal massage therapy device, which will provide the beneficial degree of heat to the affected areas.

It is a further object to provide a thermal device which may operate without the need to microwave/externally heat an operative portion thereof before its use.

It is another object of invention to provide/produce FIR electromagnetic therapy upon application of pressure against element.

It is yet further an object of invention to provide an anti-microbial, cover for direct contact with skin, thus diminishing the potential for spread of microbials. The cover and/or foam and/or pad can be treated with an anti-microbial agent.

Accordingly, there is a need for a device that can simultaneously provide both heat and manipulate soft tissue.

### BRIEF DESCRIPTION OF THE DRAWINGS

FIG. 1 illustrates a perspective view of the massage roller.

FIG. 2 is a perspective view of the massage roller.

FIG. 3 illustrates the heating element/pad.

FIG. 4 is an exemplary illustration of various electronic components of the massage roller (and/or ball, mat).

FIG. 5 illustrates a sleeve with a heating pad that can be put on a rigid shell housing the battery and the electronics.

FIG. 6 illustrates a ball with vents.

FIG. 7 illustrates a mat.

### DETAILED DESCRIPTION OF THE INVENTION

Provided is a massage roller for stretching, massage, posture, and balance. The massage roller provides benefits of thermal therapy within the therapeutic wave-lengths of Far-Infrared light.

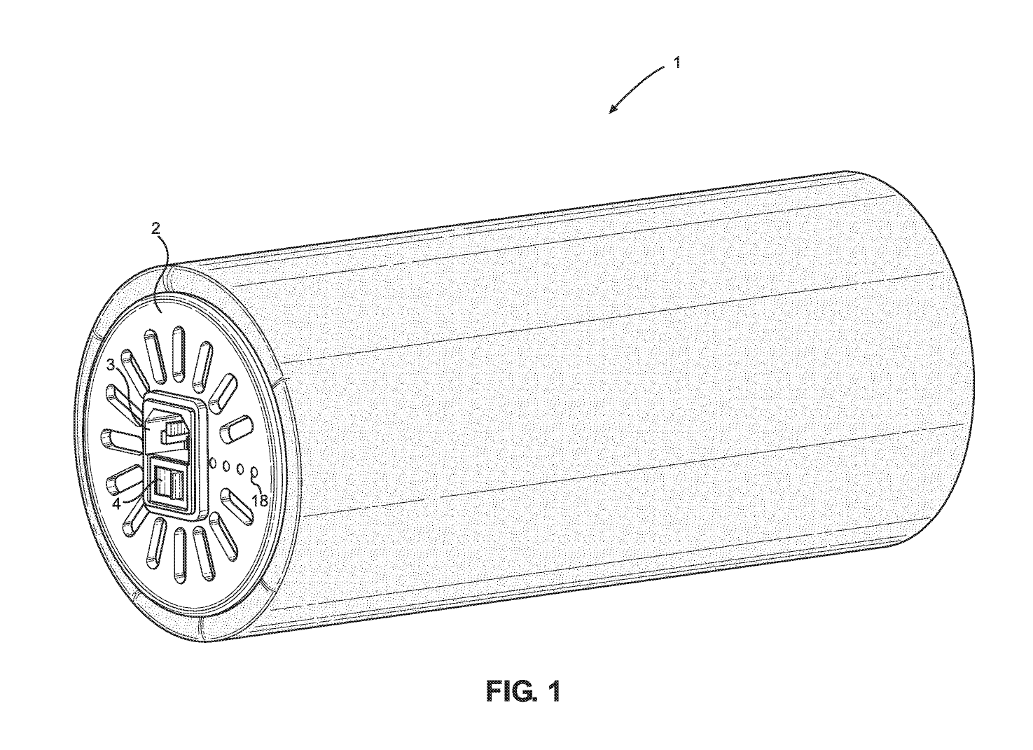

FIG. 1 illustrates a perspective view of the massage roller 1. A cylindrical body is illustrated with end cap 2. End cap 2 (and another end cap at another end of the cylindrical body) is used to close the cylindrical body that houses the electronics component and battery. End cap 2 an have vents 14 to allow for air flow. End cap 2 is a molded plastic that is affixed to the inside surface of the roller preventing access to the mounted inner components. End cap 2 can have a charging apparatus, which can include a pre-fabricated, male three-metal plug 3 and on/off switch 4. The switch 4 can be moved to the “off” position to begin the charging process and the “on” position to start flow of electric current to the heating pad. The user can plug a cord into standard wall-plug and other side into the male charging receptacle 3. The rechargeable battery 24 will start charging as indicated by end cap 2 LED lights 18. LED lights 18 can be arranged in a straight line or other configuration.

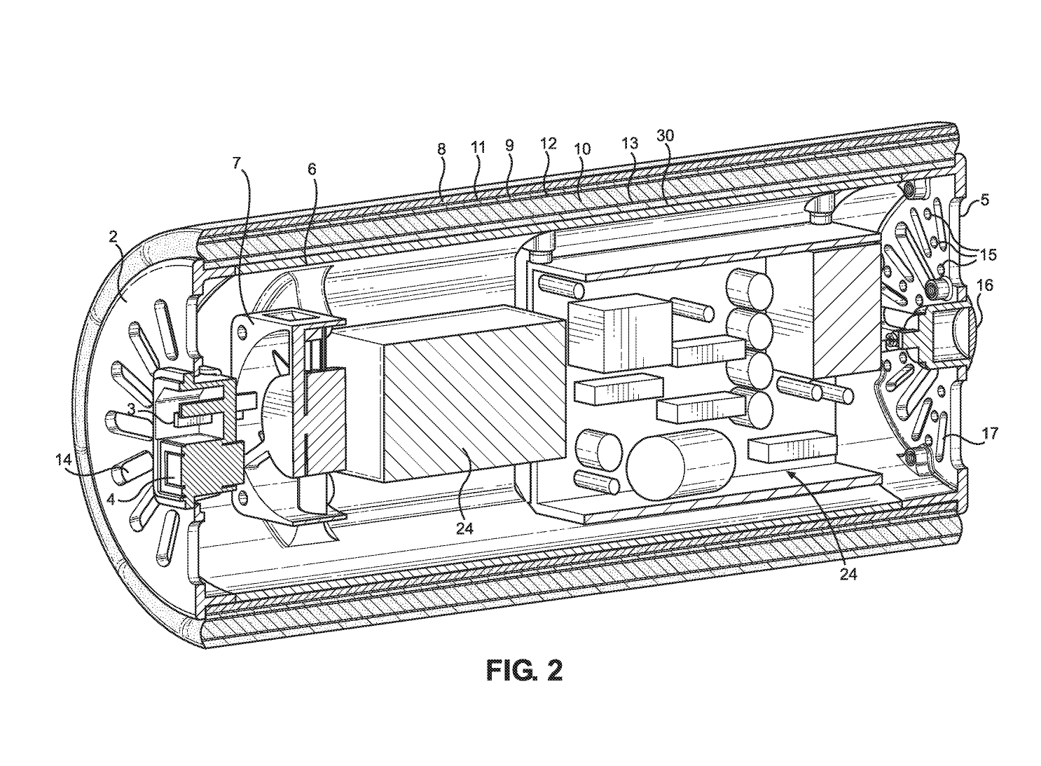

FIG. 2 illustrates a cross-section of the massage roller 1. A cylindrical shell 6 in form of a hollow shell runs along massage roller 1. The cylindrical shell 6 can be made from a rigid plastic. The term rigid means that where the structure is subjected to a force of one pound with a circular straight surface having a 1 inch diameter, the structure does not move by more than 0.3 mm. Typically, the structure does not move at all. A rechargeable battery 24 is placed inside cylindrical shell 6. A housing 14 for electronics (such as a circuit board) is also placed inside of cylindrical shell 6. A fan 7 is placed inside cylindrical shell 6 to cool the battery 24 and the electronics that are inside electronics housing 14. The hot air is moved to outside through vents 14 of end cap 2. Cylindrical shell 6 is enclosed by end cap 2 and end cap 5 on both sides.

On outside of cylindrical shell 6, support 30 is placed, which is made of a rigid material, such as plastic. Support 30 is rigid enough to withstand the weight that a person put on the massage roller 1. Over support 30 a layer of foam 10 is placed. The foam layer 10 can be attached with an adhesive to support 30 and goes around the outside of support 30. The foam layer 10 absorbs impacts from the user. The foam 10 can be a high density foam, 0.1 to 1 inch thick sheet. On top of the foam 10, a heating pad 9 can be placed. Heating pad 9 can be attached to foam 10 with adhesive 12. Cover 8 is placed on top of heating pad 9. Cover 8 can be attached to heating pad 9 through adhesive 11 or kept in place by tucking in below end caps 2 and 5 and/or by attaching its ends to inside of support 30. Cover 8 can be made from polyurethane laminate fabric.

End cap 5 is used to house LED lights 15, a heating level button 16, and ventilation openings 17. LED lights 15 are arranged as a ray-like circular pattern of yellow, orange and red colors (three rows in concentric arrangement). Circular patterned lighting 15 is utilized to indicate heating level (high, medium and low).

Battery 24 can be a lithium ion battery, wrapped in battery shrink wrap heat PVC insulation. It can be rated at 18.1v, 5S2P in its configuration, utilizing 18650 batteries. Normal capacity can be rated at ˜4.3 Ah, with an interior resistance of ˜550 mΩ. Maximum charge voltage can be 21v, minimum discharge voltage can be 13.75V; outgoing voltage can be rated at a range of 19.5V to 22V.

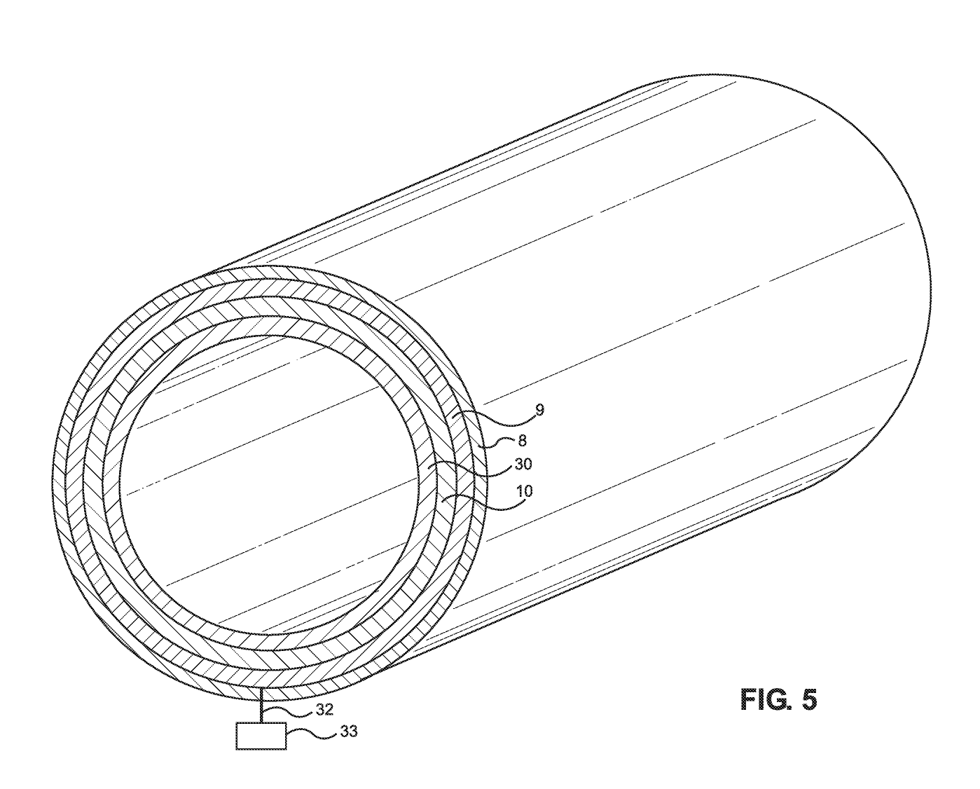

FIG. 5 illustrates a sleeve 31 that is put on cylindrical housing 6, so that a user can have his or her own personal massage roller without the electronic components. Cover 8/heating pad 9/foam 10/support 30 come as a sleeve, which as user can slid around shell 6, connect wires 32, activate, and switch to the indicated temperature. The user will then commence foam rolling on or about the patient's skin or light-loose clothing. Sleeve 31 can be fused on ends with elastic fabric to allow for expansion of sleeve 31 to fit various-other massage roller devices. At least one end of sleeve remains open, and allows for user to slide foam roller of choice into sleeve. Dimensions of sleeve can be Length: 12″-35″ and Diameter: 5.5″-9″. Port 33 for charging can also be provided.

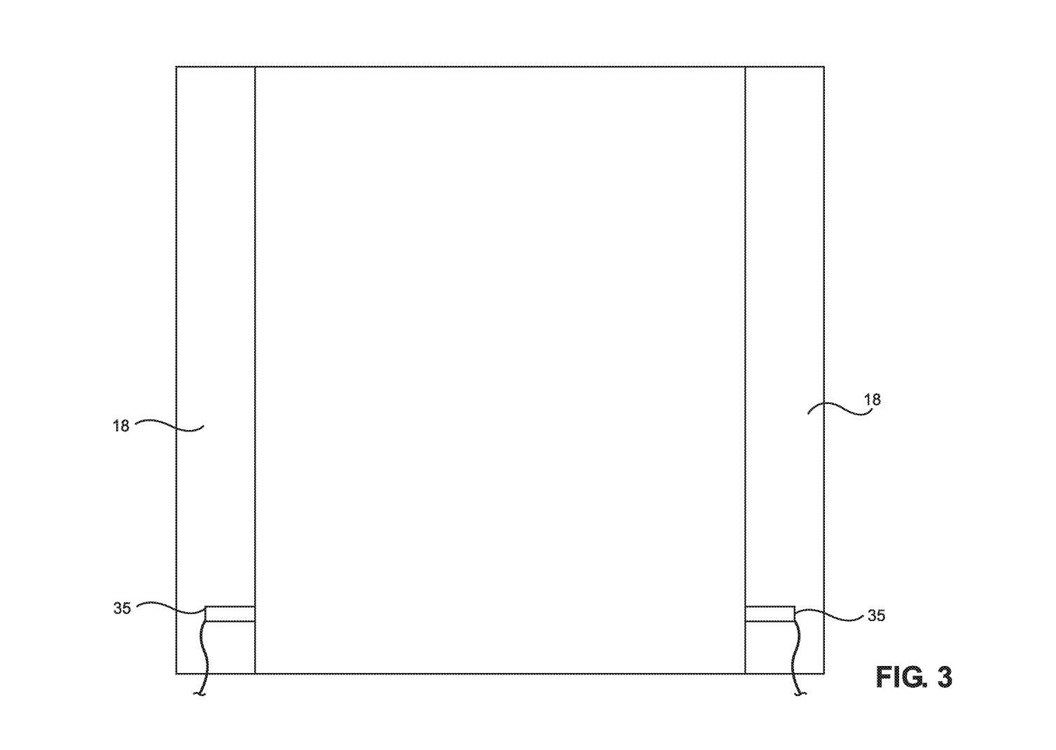

FIG. 3 illustrates heating pad 9. Heating pad 9 can be a sheet. In one embodiment a pliable, carbon fiber fabric may be employed. In this embodiment, carbon fiber (sold under the name Lamina Heat®) is woven in to a fabric with electrical wire conduit throughout. In use, the wire 35 is electrified, sending a current through the carbon fiber. The electrical resistance of carbon fiber is utilized, and ultimately the carbon is heated to a temperature up to, but not to exceed 150° F. As carbon fiber is utilized as recipient of electricity, heating element will heat evenly, and in the event of tear in fabric, heat will continue without hot-spot or risk of electrocution. The heating pad can be a rectangular sheet with two strips of copper conductors 18 placed parallel to each other on two opposite sides. Each of the two leads 35 coming from inside of shell 6 are attached to a single copper conductor 18. Electricity flows from rechargeable battery to one of the conductors, and then is forced to travel through the carbon sheet to another conductor. The resistance of the carbon to the electric flow results in a uniformly heated sheet.

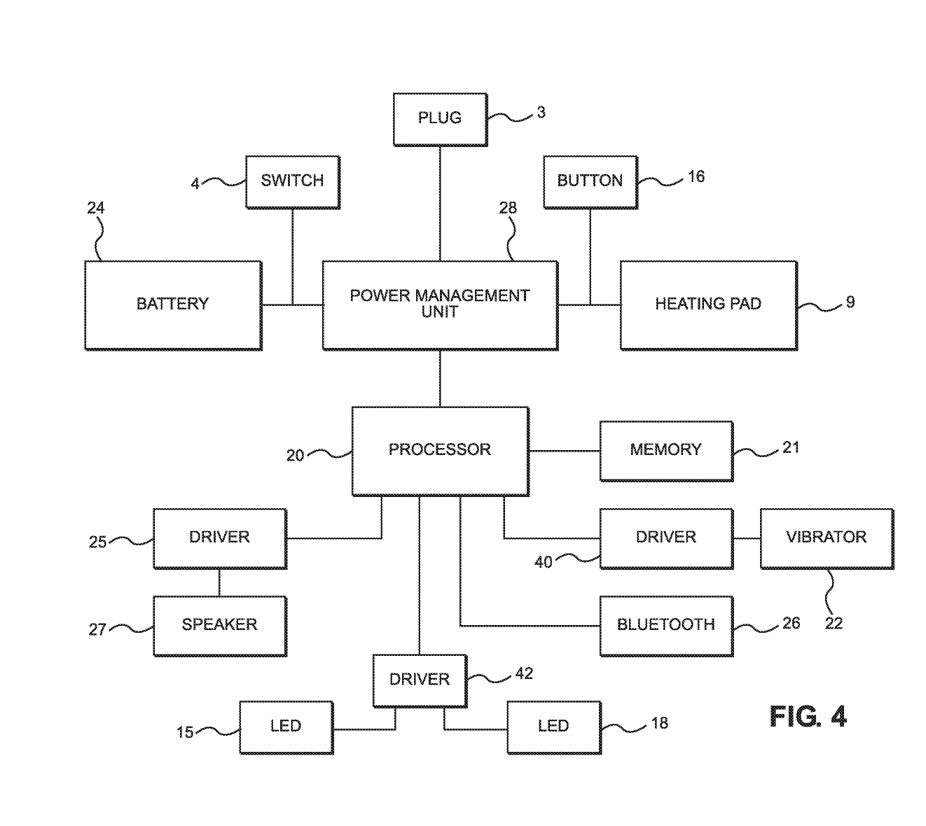

FIG. 4 illustrates exemplary electronic components of massage roller 1. The electronic components can include processer 20 for fetching and executing instructions from internal/external memory 21 and/or registers. Other electronic components can include speaker 27, vibrator 22, wireless chip (Bluetooth 26), and LED lights 15 and 18. Drivers 25, 40, and 42 cart be used to operate these components. Another component can be a power management unit 28 for controlling the operation of battery 24, including charging through plug 3, and regulating current reaching heating pad 9. Switch 4 and button 16 can be in electronic communication with power management unit 28.

FIG. 5 illustrates a ball 36. Instead of having a massage roller, the components can be configured to have the shape of a ball 36. The ball 36 can have the same components illustrated in FIG. 2, except that the components (other than the internal electronic components) can be curved to form a ball 36. There can be one or two curved caps with vents 37. Support 30 is rigid enough to withstand the weight that a person put on the massage roller 1. The ball can have a spherical rigid shell, support 30, foam 10, heating pad 9, and cover 8. Electronic components can be placed inside the ball, like a fan, battery, and a housing for other electronic components.

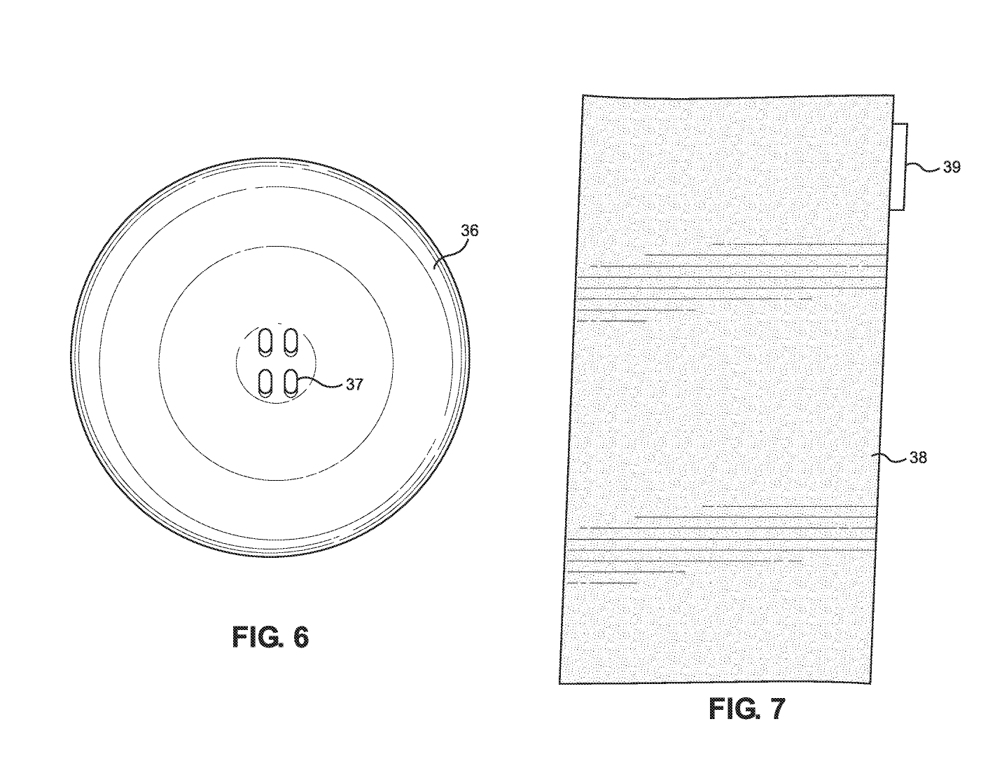

FIG. 6 illustrates a mat 38. Mat 38 can have similar components as sleeve 5, with the electronic 39 placed next to the mat. Since the mat rests on the ground, the mat may or may not have support 30. The mat can be made from layer of a foam 10, on which a heating pad 9 is placed, and cover 8.

Certain embodiments described herein are to include thermally conductive material such as carbon fiber and/or other metals/materials with high electrical conductivity.

The massage roller can provide Far Infrared (FIR) therapy. Far Infrared (FIR) (λ=3-100 μm) therapy is a subdivision of the electromagnetic spectrum that has been investigated for biological effects. It has been proven to inhibit the increase of inflammatory markers, increases blood circulation and oxygen supply to damaged tissues aiding to the reduction of chronic joint and muscle pain or sport injuries. Additionally, it induces the release of nitric oxide, which signals surrounding smooth muscle to relax, dilating the arteries, increasing blood flow and delivering oxygen and nutrients to cells in the body. Finally, FIR is associated with significantly more rapid wound healing with than without FIR. Histological findings revealed greater collagen regeneration and infiltration of fibroblasts that expressed transforming growth factor-β1 (TGF-β1) in wounds in the FIR group than in the group without FIR. Stimulation of the secretion of TGF-β1 or the activation of fibroblasts may be considered as a possible mechanisms for the promotive effect of FIR on wound healing independent of skin blood flow and skin temperature.

During use of the massage roller, the user receives the effects of both the electromagnetic spectrum of FIR, as well as the superficial effects of superficial heat within the therapeutic range of 120° f.-140° f., in addition to the benefits of foam rolling.

### REFERENCES

1. Massage roller
2. End cap a
3. Plug
4. Charge switch
5. End cap b
6. Cylindrical shell
7. Fan
8. Cover
9. Heating pad/heating element
10. Foam
11, 12, 13. Adhesive
14. Ventilation openings
15. LED light
16. Button
17. Ventilation openings
18. Conductor
20. Processor
21. Memory
22. Vibrator
24. Battery
25. Driver
26. Bluetooth
27. Speaker
28. Power Management Unit
29. Driver
30. Support
31. Sleeve
32. Wire
33. Port
35. Lead
36. Ball
37. Vents
38. Mat
39. Electronics housing for Mat
40. Driver
42. Driver

What is claimed is:

1. A massage roller or a ball comprising:
a) a rigid cylindrical or spherical body;
b) a sheet of a heating pad placed over the body;
c) a rechargeable battery placed inside of the body;
wherein current from the battery makes the heating pad increase in temperature.

2. The massage roller or ball of claim 1, wherein the heating pad gets heated to a temperature of 100 to 150 Fahrenheit.

3. The massage roller or ball of claim 1, further comprising a sheet of foam placed in between the heating pad and the cylindrical or spherical body.

4. The massage roller or ball of claim 1, further comprising two end caps, each end cap placed at one end of the cylindrical body.

5. The massage roller or ball of claim 1, further comprising ventilation opening on at least one of the end caps.

6. The massage roller or ball of claim 5, further comprising a fan inside of the cylindrical body, the fan configured to move air through the vents of one of the end caps.

7. The massage roller or ball of claim 1, further comprising a rigid shell for placing electronic components, the electronic housing placed in the shell.

8. The massage roller or ball of claim 7, wherein the rigid shell is removable.

9. The massage roller or ball of claim 4, wherein of the end caps comprises a plug configured to receive a wire that carried electricity.

10. The massage roller or ball of claim 1, further comprising a laminate on top of the heating pad.

11. The massage roller or ball of claim 10, wherein the laminate is polyurethane laminate fabric.

12. The massage roller or ball of claim 1, wherein the heating pad is made from a carbon based material

13. The massage roller or ball of claim 12, wherein the carbon based material is a sheet of woven carbon fiber.

14. The massage roller or ball of claim 1, wherein the heating pad has two strips of conductors that do not touch each other, each strip in contact with a lead wire.

15. The massage roller or ball of claim 14, wherein the heating pad is rectangular in shape, and the two strips of conductors are in parallel configuration along opposite sides of the pad.

16. The massage roller or ball of claim 1, wherein the heating pad and the cylindrical body are in form of a removable sleeve.

17. The massage roller or ball of claim 16, wherein the sleeve further comprises a cover.

18. A massage roller comprising:
a) a rigid cylindrical body;
b) two end caps, with each end cap placed at one end of the cylindrical body;
c) a sheet of foam placed over the cylindrical body;
d) a sheet of woven carbon fiber placed over the foam, the sheet of carbon having two conductors that do not touch each other;
e) a cylindrical shell placed inside of the body;
f) a rechargeable battery placed inside of the shell;
f) two lead wires carrying current from the battery to the conductor, each wire connected to one of the conductors;
wherein current from the battery heats the heating pad.

19. The massage roller or ball of claim 18, wherein the shell is removable.

20. The massage roller or ball of claim 18, further comprising a fan for moving air out of the shell from a plurality of vents on at least one of the end caps.
